(19) United States
(12) Patent Application Publication
(10) Pub. No.: US 2024/0424333 A1
(43) Pub. Date: Dec. 26, 2024

(54) ECO-FRIENDLY FIRE-FIGHTING COMPOSITION AND METHOD OF USE THEREOF

(71) Applicant: GABIO BIYOLOJIK URUNLER ANONiM SiRKETi, Istanbul (TR)

(72) Inventor: Mehmet Sinan OKAY, Besiktas (TR)

(21) Appl. No.: 18/686,177

(22) PCT Filed: Aug. 18, 2022
(86) PCT No.: PCT/TR2022/050875

§ 371 Date: Feb. 23, 2024

(30) Foreign Application Priority Data
Aug. 23, 2021 (TR) 2021/013249

(51) Int. Cl. A62D 1/00

(52) U.S. Cl. CPC A62D 1/0042

(57) ABSTRACT

The present invention relates to a biochemical fire-fighting composition for extinguishing, retarding, and preventing re-ignition of fires where the fuel contains any kind of solid, liquid, or gaseous combustible materials of class A, class B, class C, or class F fires involving essentially hydrocarbons. Said composition comprises Saccharomyces cerevisiae yeast fermentation supernatant, one or more surfactants selected from the group consisting of non-ionic surfactants and anionic surfactants, and at least one protective agent, and the same is added in small amounts into the water to be used in fire-fighting activities. Consequently, there is provided, according to the invention, a multi-purpose, efficient, and environmentally-friendly liquid concentrate formulation for fighting fires effectively.

### TECHNICAL FIELD

The present invention relates to chemical fire-fighting compositions for extinguishing, retarding, and preventing reignition of fires, particularly forest fires and fires at sea, fueled from all kinds of flammable solids, liquids and gases of organic origin.

### PRIOR ART

Fires, particularly wildfires, classified both as disasters and emergencies, are a growing natural hazard in most regions of the world, posing steadily a serious threat to life, property, and human habitation. They can rapidly burn vast areas and destroy everything, i.e. trees, homes, animals and humans in their paths. Together with the important pending problems concerning the future of our world, such as climate change and global warming, forest fires have steadily become a more pronounced threat globally.

Fire will occur when fuel, oxygen, and heat-referred to as the “fire triangle”-combine. A sufficient amount of combustible material provides the energy source as “fuel”, sufficient “oxygen”—i.e. more than 15%—is required for sustaining the combustion process, and sufficient “heat”—i.e. with a temperature of more than 260-400° C.—often provides the trigger for the fire, together with the ignition/chemical exothermic reaction as the fourth factor. The fire can be extinguished if any one of the said factors is eliminated.

Fires are classified according to the type of fuel that is burning. Class A fires involve solid combustible materials of organic origin, including forest fires, such as wood, coal, paper, grass, textiles, straw, the main characteristics of which are that they generally leave embers and ashes. Class B fires caused by the combustion of flammable liquids or materials that liquefy, such as petrol, gasoline, benzol, diesel oil, fat, kerosene, solvent, tar, paraffin, alcohols, thinner, varnish, paints, and mineral oils, the main characteristics of which are that they burn with flames without leaving embers. There exists a high risk of explosion. Sea fires of oil spills and ship-originated wastes that cause substantial environmental pollution are generally included in this class, as well.

Besides, fires caused by the combustion of flammable gases are referred to as “C class” and light metal fires are referred to as D class. The definitions of “E class” for fires involving electrical appliances and “F class” for fires involving combustible cooking media, such as hot or deep oil and grease fires in kitchens, are also encountered.

While liquid extinguishers, mostly water, and dry chemical powder extinguishers can be used against Class A fires, only dry chemical powder or foam extinguishers are used for Class B fires.

Surfactants are also known as additives to water for fighting fires, especially against Class A fires, owing to their low heat absorption property and high wetting characteristics. Likewise, Class A and Class B fires can be successfully responded by forming a foam blanket with surfactants and consequently preventing the contact of oxygen in the air with combustible materials. Various inorganic materials and/or polymers can be added into the fire-fighting composition in order to increase foam quality, effectiveness and stability of ingredients.

Conventionally, water is used for fighting forest fires, and various foaming agents, in particular, or other chemicals such as FE-13, inergen, halon, halotren or Purple K. may also be included depending on the fire types. For fires on large lands, for example, the heat absorption and the wetting capacities of the surfactants generally remained insufficient. Various solvents such as glycol ether are therefore added to the composition to increase the effectiveness of the wetting agent and Class A foaming agents, which in return causes adverse environmental impact due to increased toxicity issues and the presence of volatile organic compounds.

Likewise, foam-forming compositions containing various types and amounts of fluorochemical surfactants are commonly used especially in Class B fires involving flammable liquids. They further comprise hydrocarbon surfactants and solvents and even hydrolyzed proteins to improve their wetting characteristics. The use of high molecular weight fluoropolymers is also known to reduce their negative environmental impact. For example, patent application no. U.S. Pat. No. 6,527,970 discloses a film-forming foam fire-fighting and bioremediating composition comprising a fluorinated film-forming foam surfactant, a bioremediating bacterial component comprising at least one sporogenous nonpathogenic bacteria species, a glycol ether, and a xanthan gum, wherein the bacteria species comprise a plurality of nonpathogenic Bacillus strains chosen from B. subtilis, B. polymyxa, B. licheniformis, B. amyloliquifaciens, B. pasteurii and B. laevolacticus.

Despite the said compositions of the prior art have some advantageous fire-fighting characteristics such as high wetting capacity, durable foaming, and low volatile organic matter/polymer content, they contain fluorosurfactants and solvents or at least expose the environment to fluorine, which is still among the current environmental concerns. Likewise, such fire-fighting products, which mainly focus on the ability to form foam blankets, working on foam volume, density and stability, suggest the use of surfactants such as amine oxides and dispersants together with the aforesaid alternatives, and much more. Consequently, their uses may be limited depending on the class, diameter, and size of the fire and whether they contain water or not, as well.

Forest and sea fires, for example, where flammable hydrocarbons are present in a much larger area than a building or a ship fire, require the use of a much higher amount of fire-fighting compositions, which in turn results in various critical considerations such as production, supply, cost and transportation thereof, needs for equipment and field application (i.e. location, height, distance, etc.), resultant efficiency, and consequent environmental impact.

As a result, there exists today a need to provide multi-purpose, efficient, and environmentally-friendly compositions for fighting fires effectively regardless of the class and spread of the fire, wherein the fuels are flammable organic solid, liquid, or gas materials (of Classes A, B, C or F). The said features are also essential in terms of ensuring that forest fires, which pose a serious threat to wildlife today, and sea fires that affect marine life and the environment beyond the visible damage caused to sea life, can be successfully intervened. Moreover, it is expected that fire-fighting agents used as additives to water do not only harm the environment, but also treat the fire site and provide additional benefits to the ecosystem.

### Objects and Brief Description of the Invention

The primary object of the present invention is to provide an environmentally-friendly biochemical fire-fighting composition, to be simply used by adding it into the water, for extinguishing, retarding, and preventing reignition of fires efficiently, where the fuel essentially contains hydrocarbons or any kind of combustible materials of class A, class B, class C or class F fires involving hydrocarbons.

Another important object of the invention is to provide a biochemical fire-fighting composition without any environmentally harmful content, for use as an additive to water, for extinguishing an organically fueled fire of class A, class B, class C or class F, particularly forest fires and fires at sea, which directly reduces the ignition characteristic of the materials and eliminates the need for forming foam blanket.

Yet, another object of the invention is to provide a biochemical fire-fighting composition that restricts the formation of volatile organic compounds in the air and hydrocarbons on surfaces, breaking them down into glycerol and fatty acids, and raises the fire/ignition point of combustible materials.

In order to achieve the aforesaid objects, there is provided a cost-effective and environmentally-friendly biochemical liquid concentrate fire-fighting composition for use as an additive into the water in little amounts, for extinguishing, retarding, and preventing reignition of fires where the fuel essentially contains hydrocarbons or any kind of combustible materials of class A, class B, class C or class F fires involving hydrocarbons, which is free of fluorochemicals, and increases the efficiency and effectiveness in fire-fighting.

According to the present invention, the biochemical fire-fighting composition comprises a Saccharomyces cerevisiae yeast fermentation supernatant, one or more surfactants selected from the group consisting of non-ionic surfactants and anionic surfactants, and at least one preservative agent, added in small amounts into the water.

Said yeast supernatant can be produced from the culture of Saccharomyces cerevisiae by using conventional fermentation methods known from the prior art, preferably with substantially-reduced or eliminated enzymatic activity and bacterial presence, or can be obtained in ready-made form. Said reduction or elimination processes can be carried out via one or more applications to be selected from the group consisting of autolyzing, heat treatment, pasteurization, denaturation of cells, addition of EDTA, and subjecting them to one or more of the appropriate chromatographic conventional applications, during the production stage of the supernatant. The Saccharomyces cerevisiae yeast supernatant is present in the composition of the invention preferably at a concentration of 5% to 55%, by total weight.

Said surfactants can be selected from one or more surfactants from the group consisting of nonionic surfactants and anionic surfactants, or preferably from one or more nonionic surfactants, at a concentration of 5 to 55%, by weight. The weight ratio of anionic surfactants, if any, to the total surfactant content is at most 20%, preferably ranging from 0.1% to 10%. No cationic surfactant is available therein.

The nonionic surfactants can be selected from the group consisting of alkyl, aryl, polyethylene glycol ether, ethylene glycol, or any type of an ethylene oxide chain containing a water-soluble moiety, in particular nonylphenol ethoxylates, octylphenol ethoxylates, alkyl ethoxylates, ethoxylated amine salts, alkylphenol ethoxylates and other derivatives and mixtures thereof, having an ethoxylate moiety. Preferably, the nonionic surfactants comprise at least one to be selected from the group consisting of ethoxylated alcohol, ethoxylated aliphatic alcohol, amine oxide, alkylamine, ethoxylated alkylamine, ethoxylated alkyl phenol, alkyl polysaccharide, ethoxylated alkyl polysaccharide, and ethoxylated fatty acid. More preferably, it is selected from the group individually or in combination, consisting of ethoxylated dodecyl alcohol, ethoxylated octyl phenol, and tridecyl alcohol ethoxylate.

The anionic surfactants comprise at least one to be selected from the group consisting of alkylsulfonate, alkyldiphenyloxide disulfonate, alkylphenol polyoxyethylene ether phosphate ester and fatty alcohol polyoxyethylene ether sulfate, sodium lauryl sulfate, sodium dodecyl benzene sulfonate, sodium disulfonate, sodium dodecyl phosphate, and sodium dodecylate.

The preservative agents are preferably selected from the group consisting of sodium benzoate, imidazolidinyl urea, diazolidinyl urea, polyoxymethylene urea, quaternium-15, DMDM hydantoin, bromopol, glyoxal, sodium hydroxymethylglycinate, alkyl paraben, and glycerin, and are present in the composition at a concentration of 0.1% to 4.5%, by weight.

The pH of the fire-fighting composition, which is completed to 100% with water, is at most 7.0, preferably 2.5 to 6.5, more preferably 3.0 to 5.5. The water may herein refer to mains water, tap water, softened water, filtered water, purified water, pure water, or a combination thereof.

The composition may also comprise minor amounts of a sequestrant and/or stabilizer, i.e. EDTA, phosphonic acid, or a combination thereof.

The biochemical composition of the invention can be directly and simply added, preferably in the form of liquid concentrate of predetermined appropriate proportions, into the water to be used in fire-fighting activities. It may be applied by fogging or by means of a spray. In order to achieve the aforesaid objects, it is sufficient to provide the composition of the invention, for example, less than 10.000 cc, preferably 1.000 cc to 100 cc per tonne of water (˜1.000 liter) used in the final fire-fighting application, in other words, at a dilution ratio of 1:10 to 1:10.000 depending on the fire type and spread. In terms of feeding the same into the equipment to be used in the fire-fighting activity, intermediate dosage ranges can be arranged with calculations in accordance with the equipment conditions, provided that said ranges do not change in the final application. For example, in a fire truck where a dosing pump does not operate below 2%, the liquid concentrate composition of the invention is pre-diluted with water at an appropriate rate, and the final target ratio of the application are subsequently provided with the mixture, for cost and content savings.

The composition of the invention has no corrosive effect, is non-toxic, and is 100% naturally soluble. Owing to the physical and physicochemical advantages thereof, higher operational efficiency and higher gains with appropriate operating conditions can be obtained in fire-fighting. The supernatant obtained from S. cerevisiae yeast cells and employed in the composition content provides an unexpected technological added value since very low amounts of the same is added into water, it can also be applied in fires originating from Class B liquids, and eliminates the need for forming a foam blanket. Such use is therefore completely distinguished from the primary purposes and applications of the prior art.

The supernatant, purified from plant origin, and preferably with reduced or eliminated enzymatic activity and bacterial presence, does not contain chemical oils, pollutants, flammable substances or toxic substances since it is produced on the basis of biological extract. Together with non-ionic-anionic or preferably with non-ionic biological surfactants and other ingredients of the composition of the invention, the said advantages are enhanced synergistically. The composition rapidly breaks down hydrocarbons on the surfaces and volatile organic compounds in the air into glycerol and fatty acids, owing to the primary function of the supernatant content thereof. It destroys their flammability. It also raises the fire/ignition degree of combustible materials.

The surfactants included in the fire-fighting composition according to the invention only for increasing the diffusion, not for preventing the contact between flammable materials and the air, as other surfactants/foams of the prior art have made for trying to extinguish the fire, which is one of the reasons for failure especially in fighting sea and forest fires. The invention composition does comprise neither fluorochemicals nor bromine-containing halocarbons. It is not used in fires having the risk of electric shock (E Class).

Owing to the biochemical composition of the invention and the method of use thereof, the need for water is reduced, no harmful effect on living things, nature, and the atmosphere occurs, no pollution or health risk is observed, soil and water are refreshed, and thanks to the yeast supernatant-based bio-extract thereof, which is also a bio-nutrient, rapid restoration and revival of plants and microbiome can be achieved.

These and other features, advantages, and embodiments of the present invention will become more apparent from, and will be understood more clearly by reference to, the following detailed description and the associated examples.

### DETAILED DESCRIPTION

The use of supernatants obtained by fermentation from yeast cultures such as Saccharomyces cerevisiae, Kluyveromyces marxianus, Kluyveromyces lactis, Candida utilis, ZygoSaccharomyces, Pichia, and Hansanula together with surfactants and preservatives is known from the prior art teachings for the removal of heavy minerals and hydrocarbons in industrial wastes, organic and odorous micro-organisms and heavy metals in sewage and wastewater, biofilm formation on surfaces and pests in agriculture.

Saccharomyces cerevisiae is mainly known as baker's yeast. Named among the most well-known and best-studied important yeast strains for use in beer, wine, and bread baking since ancient times, S. cerevisiae strains are described as “generally recognized as safe” organisms by the US Food and Drug Administration (FDA), which means that these cells can be freely manipulated without public concern. The culture of S. cerevisiae included in the composition of the invention and the preparation of supernatant thereof are also well known from the prior art.

For instance, U.S. Pat. No. 3,635,797A discloses that the yeast S. cerevisiae can be initially cultured in a medium containing a sugar source such as molasses, raw sugar, soybean, or sucrose consisting of mixtures thereof. The mixture of sugar, diastatic malt, S. cerevisiae yeast, and magnesium salt is incubated for two to five days at suitable temperatures (25 to 45° C.) until the fermentation is completed, and then unwanted residues are separated, preferably by centrifugation, to obtain the supernatant. It is also mentioned the preparation of an aqueous enzymatic composition comprising an enzymatic fermentation reaction product, surfactants, citric and lactic acids, urea, and pine oil, for use in protein-surfactant systems directed to the applications of, for instance, mainly the removal of hydrocarbon-based wastes from petroleum products and industrial wastes as well as water and sewage treatment. Surfactants are selected from organic, anionic, and nonionic surfactants and inorganic alkali metal phosphates, borates, carbonates, silicates, or mixtures thereof.

The method for preparing the supernatant culture according to the invention may comprise the following basic steps: Fermenting and growing the yeast in a rich nutrient medium, then removing cells and residues by centrifugation, mixing the obtained fermentation supernatant with sodium benzoate, imidazolidinyl urea, diazolidinyl urea and/or other mentioned suitable agents, for instance, heating to 40-45° C., stirring for 1-2 hours to dissolve the components, then mixing the resulting intermediate with other ingredients, such as surfactants and preservatives, to obtain the final composition, and if necessary, adjusting the PH to 2.5. to 6.5, preferably 3.0 to 4.5.

For the inactivation and treatment of S. cerevisiae cells, various conventional methods can be applied, such as lyophilization, denaturation, pasteurization, autoclaving, irradiation, heat treatment, and chemical treatment with alkaline solutions, ethanol, formaldehyde, and acetone in order to reduce or eliminate enzymatic activity and bacterial presence therein. Likewise, an appropriate method may also be selected among the applications such as (heat) shocking via heating up to 50-70° C. for 2 to 24 hours-before or after centrifugation- and/or mechanical physical stressing (pressing, rolling, high-pressure homogenization), chemical degradation (alcohol extraction, EDTA addition, cell lysis) and/or anion exchange chromatography.

Since non-ionic surfactants are not affected by acidic or basic environments, they are the most widely used surfactants in detergent, cosmetics, and other similar industries. They are generally and effectively used in wetting, dispersing, and spreading activities and as emulsifiers, foaming (control) agents, cleaning agents for detergents, general cleaning agents, wetting agents in textile and pesticide formulations. They have compatibility with other surfactants.

In a preferred embodiment of the present invention, the final composition in concentrated liquid form comprises about 5% to 55% Saccharomyces Cerevisiae fermentation supernatant, about 5% to 55% nonionic surfactant, 1% to 7.5% anionic surfactant, about 0.1% to 3.5% sodium benzoate, and/or about 0.001% to 0.04% imidazolidinyl urea and/or about 0.01% to 0.4% diazolidinyl urea, by weight. Also, a nitrogen source such as urea or ammonium nitrate can be added at a concentration of 3% to 30% by weight in the final composition. If necessary, the pH of said composition can be adjusted with an acid such as citric acid or phosphoric acid to a maximum of 7.0, preferably 3.0 to 6.5.

As an example of another preferred embodiment, the composition of the invention comprises, by weight, 25.0% S. cerevisiae supernatant, 23.75% ethoxylated octyl phenol, 1.25% alkyldiphenyloxide disulfonate, 0.20% diazolidinyl urea, 0.35% sodium benzoate, and water to add up to 100%.

Depending on the fire type (i.e. forest fire, crude oil fire at sea) and type of the water (i.e. sea water, lake water, well water, main water) to be used thereagainst, said liquid concentrate composition may be provided with a dilution ratio of less than 1:10, preferably 1:100 to 1:10.000, per tonne of water (˜1000 litre) to be used for fighting fires, by volume. This use, for example, is decreased to a dilution ratio of about 1:10 per tonne of water, in ship engine room fires that require more intense concentrations. The said applications do not prevent breathing and are harmless to human health and the environment. In fact, since it reduces the toxic effect of smoke, it provides an easy and effective response to fire in closed areas.

While certain examples and embodiments of the present invention have been described so far, it is apparent to those skilled in the art that various other changes and modifications can be made without departing from the spirit and scope of the invention. Therefore, with the attached claims, it is ensured that such changes and modifications are included in the scope of protection without departing from the scope and integrity of the invention.

1-11. (canceled)

12. A biochemical liquid concentrate fire-fighting composition, free of fluorochemical substances, for use simply as an additive into the water with a dilution ratio of less than 1:10 by volume, for extinguishing, retarding and preventing re-ignition of fires, where the fuel essentially contains hydrocarbons or any kind of combustible materials of class A, class B, class C or class F fires involving hydrocarbons, comprising
Saccharomyces cerevisiae yeast fermentation supernatant at a concentration of 5 to 55%, by weight;
one or more surfactants selected essentially and only from nonionic surfactants at a concentration of 5 to 55%, by weight; and
at least one preservative agent selected from the group consisting of sodium benzoate, imidazolidinyl urea, diazolidinyl urea, polyoxymethylene urea, quaternium-15, DMDM hydantoin, bromopol, glyoxal, sodium hydroxymethylglycinate, alkyl paraben, and glycerin, at a concentration 0.1% to 4.5%, by weight;
wherein said yeast supernatant produced by fermentation from the Saccharomyces cerevisiae culture lacks any enzymatic activity or bacterial presence.

13. The composition according to claim 12, wherein said nonionic surfactant content comprises at least one surfactant selected from the group consisting of amine oxide, ethoxylated alcohol, ethoxylated aliphatic alcohol, alkylamine, ethoxylated alkylamine, ethoxylated alkyl phenol, alkyl polysaccharide, ethoxylated alkyl polysaccharide, and ethoxylated fatty acid.

14. The composition according to claim 13, wherein said nonionic surfactant content comprises at least one surfactant selected from the group consisting of ethoxylated dodecyl alcohol, ethoxylated octyl phenol, and tridecyl alcohol ethoxylate.

15. The composition according to claim 12, further comprising a nitrogen source having urea or ammonium nitrate at a concentration of 3 to 30%, by weight.

16. The composition according to claim 12, further comprising EDTA, phosphonic acid, or a combination thereof as a sequestrant and/or stabilizer.

17. The composition according to claim 12, characterized by having a pH below 7.0, preferably between 2.5 and 6.5.

18. A method for extinguishing, retarding and preventing re-ignition of fires where the fuel essentially contains hydrocarbons or any kind of combustible materials of class A, class B, class C, or class F fires involving hydrocarbons, comprising use of the liquid concentrate biochemical composition according to claim 12, as an additive into the water characterized in that the said liquid concentrate biochemical composition is added into water to be used for fighting fires with a dilution ratio of 1:100 to 1:10.000, per tonne of water (˜1000 litre), by volume.

19. A biochemical liquid concentrate fire-fighting composition comprising
about 5% to 55% Saccharomyces cerevisiae fermentation supernatant comprising;
a Saccharomyces cerevisiae yeast fermentation supernatant at a concentration of about 5 to 55% nonionic surfactant; and
about 1% to 7.5% anionic surfactant, by weight;
wherein said yeast supernatant produced by fermentation from the Saccharomyces cerevisiae culture lacks any enzymatic activity or bacterial presence; and
wherein the biochemical liquid concentrate fire-fighting composition can be used simply as an additive into water, for extinguishing, retarding and preventing re-ignition of fires;
wherein the fuel essentially contains hydrocarbons or any kind of combustible materials of class A, class B, class C or class F fires involving hydrocarbons; and
wherein the fire is free of fluorochemical substances.

20. The composition according to claim 19, further comprising at least one preservative agent selected from the group consisting of sodium benzoate, imidazolidinyl urea, diazolidinyl urea, polyoxymethylene urea, quaternium-15, DMDM hydantoin, bromopol, glyoxal, sodium hydroxymethylglycinate, alkyl paraben, and glycerin, at a concentration 0.1% to 4.5%, by weight.

21. The composition according to claim 20, wherein the preservative is about 0.1% to 3.5% sodium benzoate, and/or about 0.001% to 0.04% imidazolidinyl urea and/or about 0.01% to 0.4% diazolidinyl urea, by weight.

22. The composition according to claim 19, further comprising a nitrogen source having urea or ammonium nitrate at a concentration of 3 to 30%, by weight.

23. The composition according to claim 19, characterized by having a pH below 7.0, preferably between 3.0 and 6.5.

24. The composition according to claim 23, wherein the pH of the composition can be adjusted with an acid such as citric acid or phosphoric acid.

25. The composition according to claim 19, wherein the liquid concentrate biochemical composition is an additive added into water to be used for fighting fires with a dilution ratio of 1:100 to 1:10.000, per tonne of water (˜1000 litre), by volume.
